(19) United States
(12) Patent Application Publication
(10) Pub. No.: US 2021/0386894 A1
(43) Pub. Date: Dec. 16, 2021

(54) UV Sensing and Disinfecting System and Virus Tracking System and Associated Methods

(71) Applicant: Todd Bracher Studio LLC, Brooklyn, NY (US)

(72) Inventors: Todd Bracher, Brooklyn, NY (US); Tina Michelle Cheng, Brooklyn, NY (US)

(73) Assignee: Todd Bracher Studio LLC, Brooklyn, NY (US)

(21) Appl. No.: 17/303,727

(22) Filed: Jun. 7, 2021

Related U.S. Application Data
(60) Provisional application No. 62/705,073, filed on Jun. 10, 2020.

(51) Int. Cl. A61L 2/24; A61L 2/10; A61L 2/28

(52) U.S. Cl. CPC A61L 2/24; A61L 2/10; A61L 2202/14; A61L 2202/11; A61L 2/28

(57) ABSTRACT

A sterilizing system including a sterilization device that emits UVC radiation and visible radiation into an irradiation field, a sensor to detect an object within the irradiation field and measure photoluminescence of a target pathogen within the irradiation field, and an indicator device. Control circuitry may control operation of sterilizing radiation responsive to detecting the target pathogen and collect information about contamination levels. A server may collect and aggregate contamination level information from multiple sterilization devices.

### FIELD OF THE INVENTION

The present invention relates to systems and methods for sterilizing systems for target pathogens and tracking the detection of target pathogens.

### BACKGROUND

Irradiation of surfaces with UV light is a common disinfection method. However, developments in UVC radiation that are effective in disinfecting against target pathogens have enabled new opportunities in sterilization involving human tissue. Systems that can sterilize human tissue that is a common vector for communicable pathogens, such as the hands, are needed to prevent the spread of such pathogens. Moreover, the targeting of such pathogens presents an opportunity for collecting important epidemiological information at the time of UV disinfection. Accordingly, there is a need in the art for a system that can both disinfect surfaces to remove target pathogens while also collecting and aggregating epidemiological information about such pathogens.

This background information is provided to reveal information believed by the applicant to be of possible relevance to the present invention. No admission is necessarily intended, nor should be construed, that any of the preceding information constitutes prior art against the present invention.

### SUMMARY

An embodiment of the invention is directed to a sterilizing system comprising a sterilization device. The sterilizing device comprises a housing, a plurality of radiation-emitting devices carried by the housing, comprising, a first radiation-emitting device configured to emit electromagnetic radiation into an irradiation field having a peak intensity wavelength within a wavelength range from 217 nm to 227 nm, and a second radiation-emitting device configured to emit electromagnetic radiation having a peak wavelength within a visible spectrum, into the irradiation field to indicate the location of the irradiation field. The sterilizing device further comprises a sensor configured to detect the presence of an object within the irradiation field and measure photoluminescence of a target pathogen within the irradiation field. The sterilizing device may further comprise a dispersing apparatus configured to disperse a photoluminescent substance into the irradiation field, the photoluminescent substance being configured to luminesce when attached to the target pathogen and irradiated by radiation emitted by the plurality of radiation-emitting devices and an indicator device configured to indicate a ready status, a treatment progress status, and a treatment complete status. The sterilizing device may further comprise control circuitry coupled to each of the plurality of radiation-emitting devices, the sensor, the dispersing apparatus, and the indicator device and configured to operate the indicator device to indicate a ready status, receive an indication from the sensor indicating the presence of an object within the irradiation field, operate the second radiation-emitting device to indicate the location of the irradiation field, operate the dispersing apparatus to disperse the photoluminescent substance within the irradiation field responsive to the indication indicating the presence of an object within the irradiation field, operate the first radiation-emitting device a first time to emit radiation, receive an indication from the sensor indicating the contamination level of the object within the irradiation field, determine an radiation dosage responsive to the indication of the contamination level of the object within the irradiation field, operate the first radiation-emitting device to emit radiation to deliver the determined radiation dosage, operate the indicator device to indicate the treatment progress status as the first radiation-emitting device operates, and upon the first radiation-emitting device emitting the determined radiation dosage, operate the indicator device to indicate the treatment complete status. The sterilizing device may further comprise a communication device coupled to the control circuitry and configured to communicate across a network and transmit presence and contamination level information from the control circuitry across the network. The sterilization system may further comprise a server positioned in communication with the communication device across a network and configured to receive the presence and contamination level information, the server comprising a sterilization device database configured to store individually identifiable information about the sterilization device, and a pathogen detection database configured to store target pathogen detection and contamination level information received from the sterilization device. The server is configured to generate real-time information about the geographic distribution of the target pathogen responsive to the target pathogen detection and contamination level information stored in the pathogen detection database.

### BRIEF DESCRIPTION OF THE DRAWINGS

FIG. 1 is a perspective view of a sterilizing system according to an embodiment of the invention.

FIG. 2 is a schematic view of the sterilizing system of FIG. 1.

FIG. 3 is a perspective view of a sterilizing system according to an embodiment of the invention.

FIG. 4 is a perspective view of a sterilizing system according to an embodiment of the invention.

FIG. 5 is a perspective view of a sterilizing system according to an embodiment of the invention.

FIG. 6 is a schematic view of a pathogen detecting, sterilizing, and tracking system according to an embodiment of the invention.

FIGS. 7A-B are front and rear elevation views of a sterilizing system according to an embodiment of the invention.

FIG. 8 is a rear perspective view of a sterilizing system according to an embodiment of the invention attached to an article of personal protective equipment.

FIG. 9 is a front elevation view of a sterilizing system according to an embodiment of the invention for an automated teller machine (ATM).

FIGS. 10A-B are front and side elevation views of a sterilizing system according to an embodiment of the invention.

FIGS. 11A-B are views of a sterilizing system for door handles according to an embodiment of the invention.

FIGS. 12A-C are views of a sterilizing system incorporated into hand drying devices according to embodiments of the invention.

FIGS. 13A-E are views of a sterilizing system incorporated into human sanitation facilities according to an embodiment of the invention.

FIG. 14 is a perspective view of a sterilizing system integrated into a task light according to an embodiment of the invention.

FIG. 15 is a perspective view of a sterilizing system incorporated into a gasoline pump according to an embodiment of the invention.

FIG. 16 is a perspective view of a sterilizing system integrated into a gaming device according to an embodiment of the invention.

FIG. 17 is a perspective view of a sterilizing system according to an embodiment of the invention.

FIG. 18 is an elevation view of a sterilizing system associated with a keyboard according to an embodiment of the invention.

FIG. 19 is a perspective view of a sterilizing system integrated into a keypad according to an embodiment of the invention.

FIG. 20 is a perspective view of a sterilizing system integrated into a currency drawer according to an embodiment of the invention.

FIG. 21 is a perspective view of a sterilizing system integrated into a fan device according to an embodiment of the invention.

FIG. 22 is a front elevation view of a sterilizing system integrated into an ATM according to an embodiment of the invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a system for detecting a viral presence on a surface, determining a level of viral presence on the surface, and operating a sterilizing electromagnetic radiation (EMR) responsive to the determined level of viral presence on the surface. In some embodiments, the system may further transmit information about the detected viral presence to a viral mapping system.

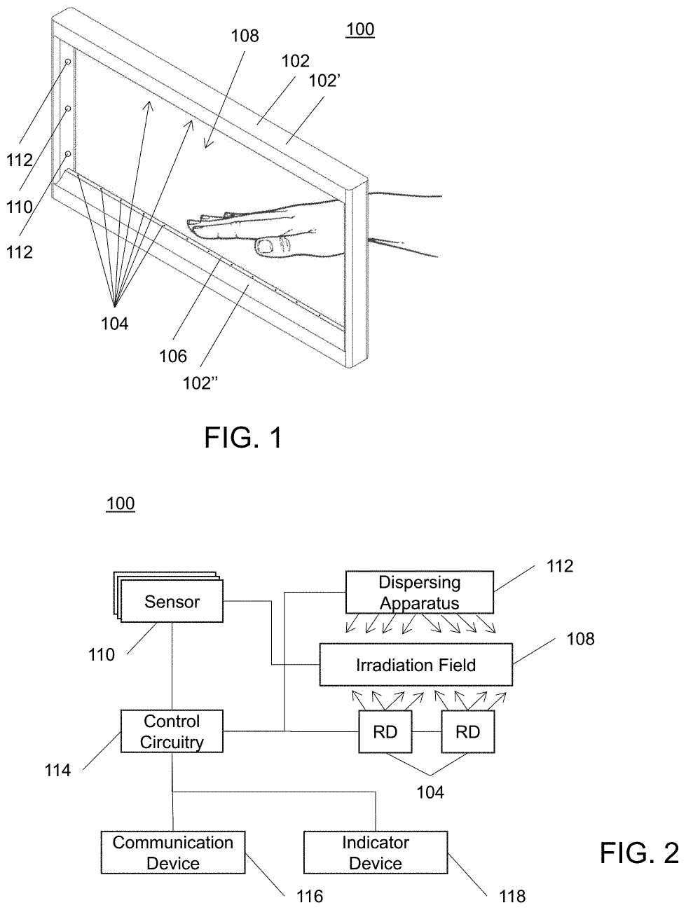

Referring now to FIG. 1, a sterilizing system 100 according to an embodiment of the invention is presented. The sterilizing system 100 may comprise a housing 102 and a plurality of radiation-emitting devices 104. The radiation-emitting devices 104 may be positioned upon and carried by the housing 102. The radiation-emitting devices 104 may be configured to emit radiation to deactivate target pathogens within the field of irradiation. Such radiation may be within specific wavelength ranges and having a specific wavelength with a maximum intensity of radiation emitted by the radiation-emitting device. In some embodiments, the radiation-emitting devices 104 may be configured to emit electromagnetic radiation having a peak intensity within the UV-C range, i.e. within a range from 200 nanometers (nm) to 280 nm. In some embodiments, the radiation-emitting devices 104 may be configured to emit electromagnetic radiation having a peak intensity within a wavelength range from 217 nm to 227 nm. In some embodiments, the radiation-emitting devices 104 may be configured to emit electromagnetic radiation having a peak intensity of 222 nm. Radiation within this range may be emitted when it is determined by the sterilizing system 100 that a living subject that is not that target of the radiation (e.g. humans, canines, felines, etc.) may be irradiated. In some embodiments, the radiation-emitting devices 104 may further be configured to emit a second electromagnetic radiation having a peak intensity within a range from 249 nm to 259 nm, and in further embodiments to emit electromagnetic having a peak intensity of 254 nm, when the sterilizing system 100 determines a living subject that is not the target of the radiation will not be irradiated. The radiation-emitting devices 104 may comprise any device operable to emit radiation within the above-described electromagnetic radiation ranges, including, but not limited to, light-emitting diodes (LEDs), laser diodes (LDs), mercury vapor discharge devices, and the like.

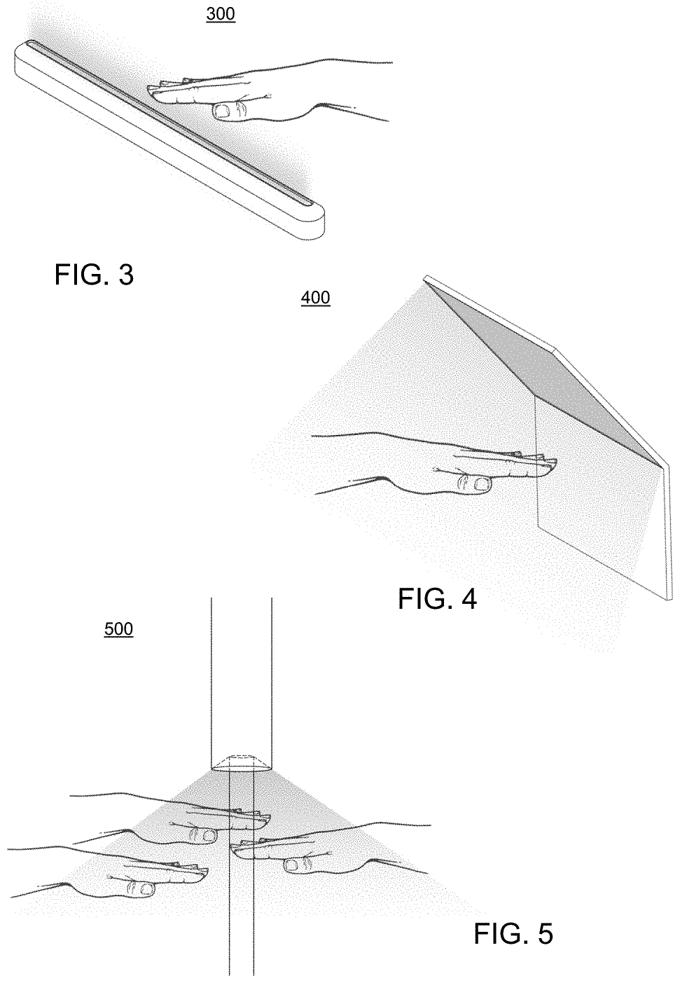

In some embodiments, the plurality of radiation-emitting devices 104 may be distributed to create an irradiation field 108 of the sterilizing system 100. The housing 102 may be configured to facilitate the positioning of the plurality of radiation-emitting devices 104 to irradiate the irradiation field 108. In the present embodiment, the housing 102 has four sides and is configured to facilitate the attachment of the plurality of radiation-emitting devices 104 to upper and lower members 102′, 102″ of the housing 102. This configuration is exemplary only and other configurations are contemplated and included within the scope of the invention, such as embodiments 300, 400, 500 seen in FIGS. 3-5.

In some embodiments, the plurality of radiation-emitting devices 104 may further comprise one or more radiation-emitting devices operable to emit radiation within the visible spectrum, i.e. having a peak wavelength within a range from 380 nm to 750 nm, defined as a second radiation-emitting device, where a first radiation-emitting device emits radiation within the UVC spectrum described above. The emission field of the second radiation-emitting device may be generally co-extensive with the irradiation field 108. By emitting radiation within the visible spectrum, the second radiation-emitting device may indicate the irradiation field 108 to the user, so that the user may be confident that the surface they desire to have target pathogens targeted is irradiated.

In some embodiments, the sterilizing system 100 may further comprise a light distributing structure 106 configured to alter the distribution of radiation emitted by the plurality of radiation-emitting devices 104. Such distribution of light may include refraction, diffusion, and collimation. Such a light distributing structure 106 may comprise one or more of a lens, a light diffuser, and other refracting structures. In the present embodiment, a light diffuser 106 comprises one or more elongate light diffusers.

The sterilizing system 100 may be configured to identify potential irradiation targets. Such identification may be accomplished by any means or method as is known in the art. In the present embodiment, the sterilizing system 100 may comprise one or more sensors 110. The sensors 110 may be configured to detect the presence of an object within the irradiation field 108. Sensors of any type as are known in the art are contemplated and included within the scope of the invention, including optical sensors such as photoconductive devices, photovoltaics, photodiodes, and phototransistors.

It is contemplated and included within the scope of the invention that any object may be positioned within the irradiation field 108 and detected by the sensors 110. In some embodiments, the sensors 110 may be configured to differentiate between living and non-living objects. As shown, a hand of a user may be positioned within the irradiation field. The sensors 110 may be operable to sense the living tissue of the hand, or any other body part, and operate the plurality of radiation-emitting devices 104 responsive to sensing living tissue. Similarly, the sensors 110 may be operable to sense non-living objects and operate the plurality of radiation-emitting devices 104 responsive thereto. Such detection may be based on detection of infrared radiation emanating from the living tissue, the sensors 110 comprising optical cameras and software operable to identify body parts within the field of view of the optical cameras, and all other means and methods of identification of living tissue as is known in the art, as well as differentiate between living and non-living objects.

The sensors 110 may be configured to detect the presence of a target pathogen on a surface within the irradiation field 108 and/or in the air of the irradiation field 108. The presence of the target may be detected by any means or method as is known in the art, including, but not limited to, photoluminescence. In the present embodiment, one or more of the radiation-emitting devices of the plurality of radiation-emitting devices 104 may be configured to emit electromagnetic radiation configured to cause the target pathogen, or a substance attached, bonded, or otherwise coupled thereto, to luminesce. In some embodiments, the sterilization system 100 may further comprise a dispersing apparatus 112 configured to disperse a photoluminescent substance configured to attach to the target pathogen within the irradiation field 108. The photoluminescent substance may comprise adenosine triphosphate (ATP). The sterilizing system 100 may be configured to detect the presence of an object within the irradiation field 108 using the sensor 110 and activate the dispersing apparatus 112 to disperse the photoluminescent substance into the irradiation field to coat the outer surface of the object. The sterilizing system 100 may then operate the plurality of radiation-emitting devices 104 to emit radiation configured to cause the photoluminescent substance that has binded to the target pathogen to luminesce, such luminescence being detected by the sensor 110. If the luminescence is detected, the sterilization system 100 may operate the plurality of radiation-emitting devices 104 to emit sterilizing radiation as described above.

Moreover, the intensity, dispersal, and other characteristics of the luminescence may be detected by the sensor 110 may be detected to determine a level of contamination of the target pathogen. The sterilizing system 100 may further be configured to vary at least one of the intensity of emission and the length of irradiation duration of the plurality of radiation-emitting devices 104 responsive to the determined level of contamination, with comparatively higher levels of contamination resulting in at least one of an increase in the intensity of radiation emitted and lengthening of the irradiation duration.

Furthermore, the sterilizing system 100 may be operable to periodically operate the dispersing apparatus 112 without the presence of an object being detected within the irradiation field 108 such that the photoluminescent substance may bind with the target pathogen in the air of the irradiation field 108 and then operate the plurality of radiation-emitting devices 104 to emit radiation configured to cause the photoluminescent substance that has binded to the target pathogen to luminesce, such luminescence being detected by the sensor 110. If the luminescence is detected, the sterilization system 100 may operate the plurality of radiation-emitting devices 104 to emit sterilizing radiation as described above.

Referring now specifically to FIG. 2, additional elements of the sterilizing system 100 will be discussed. The sterilization system 100 may further comprise control circuitry 114. The control circuitry 114 may be positioned in electrical communication with the radiation-emitting device 104 and configured to provide power to and control the operation of the radiation-emitting device 114. The control circuitry 114 may include a processor device, such as a microprocessor, an integrated circuit (IC), a field programmable gate assemblies (FPGA), and the like. The control circuitry 114 may further comprise memory in communication with the processor device, operable to have software and other data stored therein and be readable and writable by the processor device. The control circuitry 114 may further comprise a communication device 116 operable to communicate with a remote computerized device either directly or across a network, including personal area networks (PANs), local area networks (LANs), wide area networks (WANs), and any other network as is known in the art, including devices such as wired communication devices that include universal serial buses (USBs), Ethernet devices, and the like, as well as wireless communication devices operable to communicate using any wireless communication standard, including all IEEE 802 standards, including Wi-Fi, Bluetooth, Bluetooth LE, and the like. For example, the communication device 116 may be one or more of a Wi-Fi networking card, a Bluetooth device, and an Ethernet card.

The control circuitry 114 may be configured to operate the plurality of radiation-emitting devices 104, the sensors 110, and the dispersing apparatus 112 as described hereinabove. The control circuitry 114 may be configured to operate the sensors 110 to detect the presence or absence of an object within the irradiation field 108, interpreting signals generated by the sensors 110 to make such a determination. The control circuitry 114 may further be configured to operate the dispersing apparatus 112 and the plurality radiation-emitting devices 104 to emit radiation to cause the luminescence of the photoluminescence of the substance dispersed by the dispersing apparatus 112 into the irradiation field 108 and operate the sensors 110 to detect, and interpret the signals generated by the sensors 110, to determine the presence of the target pathogen within the irradiation field 108, and operate the plurality of radiation-emitting devices 104 to emit sterilizing radiation in the UVC range as described hereinabove. Moreover, the control circuitry 114 may be configured to determine a radiation dosage responsive to the contamination level sensed by the sensors 110, with increasing contamination levels being associated with increased radiation dosages. The control circuitry 114 may further be configured to operate the plurality of radiation-emitting devices 104 responsive to the determined radiation dosage to irradiate the surface with the determined radiation dosage, including varying the intensity and duration of irradiation.

In some embodiments, the sterilization system 100 may further comprise an indicator device 118. The indicator device 118 may be operable to indicate a status of the sterilization system to the user. The indicator device 118 may comprise one or more devices to communicate with the user, including, but not limited to, visual communication, including the user of LEDs, LCD displays, OLED displays, and the like, and audio communication, including a speaker, a piezo device, and the like. Furthermore, the control circuitry 114 may be configured to operate the indicator device 118 to convey the status of the sterilization system 100. In some embodiments, the control circuitry 114 may be configured to operate the indicator device 118 to indicate a ready status, indicating the sterilization system 100 is ready to sterilize. Such a ready status may comprise displaying the word “READY” or similar sentiment on a display of the indicator device 118, emitting light within a first wavelength range corresponding to a first color, such as green, by operating a radiation emitting device of the indicator device 118, and operating a speaker of the indicator device 118 to emit a first audible indication of a ready status. The control circuitry 114 may further be configured to operate the indicator device 118 to indicate a treatment progress status, indicating treatment (irradiation) is in progress and, in some instances, the level of completion of treatment. Such a treatment progress status may comprise the control circuitry 114 operating a display device of the indicator device 118 to display at least one of a time remaining for treatment, a percent completion of treatment, and a textual and/or graphical representation that irradiation is ongoing. A treatment progress status may comprise the control circuitry 114 operating a radiation-emitting device of the indicator device 118 to emit electromagnetic radiation within a second wavelength range associated with a second color that is different from the first color, such as yellow or amber. A treatment status indicator may comprise the control circuitry 114 operating a speaker of the indicator device 118 to emit a second audible indication that is different from the first audible indication to indicate a treatment in progress status. The control circuitry may further be configured to operate the indicator device 118 to indicate a treatment complete status. Such a treatment complete status may comprise operating a display of the indicator device 118 to give a textual and/or graphical indication of treatment being complete, operating a radiation-emitting device of the indicator device 118 to emit electromagnetic radiation within a wavelength range associated with a third wavelength range associated with a third color, that is different from the second color and may be the same as or different from the first color, and operating a speaker of the indicator device to emit a third audible indication different from the second audible indication and may be the same as or different from the first audible indication to indicate a treatment complete status.

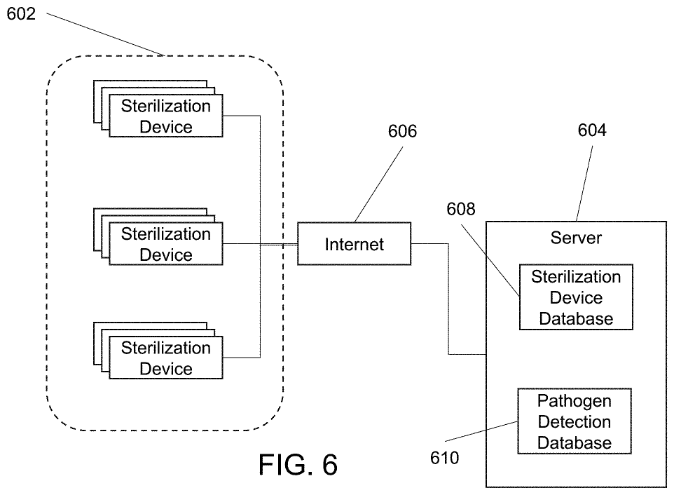

Referring now to FIG. 6, another aspect of the inventions is presented. In the present embodiment, a pathogen sterilization and tracking system according to an embodiment of the invention is presented. The system may comprise a plurality of sterilization systems 602 as described hereinabove, each sterilization system being operable to detect the presence and level of contamination of the target pathogen and emit sterilizing radiation responsive thereto. Moreover, each of the sterilizing systems may be configured to communicate with a server 604 to communicate the detection and level of contamination of the target pathogen. The plurality of sterilization systems 602 may communicate with the server 604 via any network as is known in the art, including, but not limited to, the Internet 606.

The server 604 may be a server as is known in the art include all necessary components necessary to communicate with the plurality of sterilization systems 602 via the Internet 606, including a network communication card, as well as a processor and memory as described above to receive and process information from the plurality of sterilization systems 602. The server 604 may comprise a sterilization system database 608 configured to individually identify each sterilization system of the plurality of sterilization systems 602. and the geographic location of each sterilization system.

The server 604 may further comprise a pathogen detection database 610. The pathogen detection database 610 may comprise all information regarding the detection of pathogens by the plurality of sterilization systems 602. Such information may include the detection of the target pathogen and the level of contamination. This information may be correlated with the geographic location of the sterilization system from which the pathogen detection information was received. Such correlation may be added to the pathogen detection database to provide information regarding the geographic distribution of the target pathogen. From the data comprised by the pathogen detection database, real-time analyses regarding the spread and level of infection for pandemics associated with the target pathogen. Such data may facilitate the identification of transmission and spread of the target pathogen, increases or decreases in the extent of infection, and generation of graphical representations of this information, such as heat maps. The server 604 may be configured to process the data and identify patterns regarding the detection of the target pathogen and identify transmission patterns thereof, as well as generate graphical representations.

In some embodiments, the sterilization system database 608 and the pathogen detection database 610 may be incorporated into the sterilization system 602. This advantageously provides the sterilization system 602 with the ability to emit a particular wavelength based on identification of a pathogen by the device without the need to maintain internet connectivity.

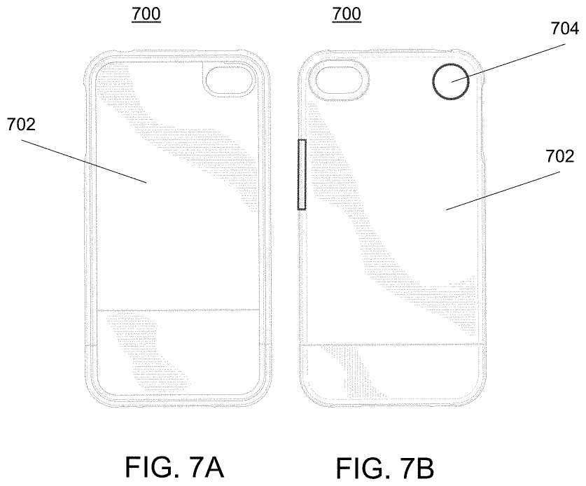

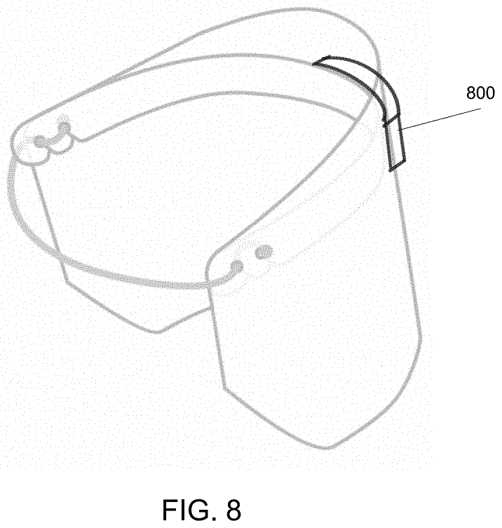

Referring now to FIGS. 7A-B, another embodiment of a sterilization system 700 according to an embodiment of the invention is presented. The sterilization system 700 may comprise a housing 702 configured to be positioned around a personal electronic device, such as a smartphone. The housing 702 may be configured to act as a case, such as a protective case, for the smartphone. The sterilization system 700 may further comprise an integrated sensing and emitting apparatus 704. The integrated sensing and emitting apparatus 704 may be operable to emit phosphorescing and sterilizing radiation as described hereinabove. Furthermore, the sterilization system 700 may be positioned in communication with the device to which it is attached, by one or both of a physical connection with a physical port of the smartphone, such as USB, or by wireless communication, such as by Bluetooth, and all other communication standards and methods as described hereinabove. In such embodiments, the sterilization system 700 may comprise a communication device operable to communicate as described. This will also allow access to the sterilization system 702 of data on the smartphone or other personal electronic device. This data may, for example, include location data, calendar data, or any other type of data that may be accessible from such a personal electronic device. This data may be used by the sterilization system 702 to emit a particular wavelength to achieve sterilization.

In some embodiments, a radiation-emitting device and a sensor as described hereinabove may be incorporated into a smartphone device. In such embodiments, the operation of the radiation-device and the sensor may be controlled by software running on the smartphone, such that detection and irradiation of target pathogens may be performed entirely by the smartphone. Moreover, data generated by the sensor may be transmitted to a pathogen-tracking server as described above using communication capabilities built into the smartphone.

Referring now to FIG. 8, another embodiment of the invention is presented. In the present embodiment, the sterilization system 800 may be substantially as described above, including radiation-emitting devices, sensors, and dispersing apparatus, as well as necessary controlling and communication componentry. The sterilization system 800 may be configured to be attached to personal protective equipment, such as a face shield. The sterilization system 800 may be configured to sterilize the face shield, particularly the outward-facing portion of the face shield. While a face shield is shown, any type of PPE is contemplated and included within the scope of the invention as having the sterilization system 800 attached thereto and sterilized thereby. It is also contemplated that the sterilization system 800 may be configured to also be inwardly facing to provide sterilization to the user or wearer of the PPE, but be configured in a manner that does not interfere with routine activities of the wearer while in use.

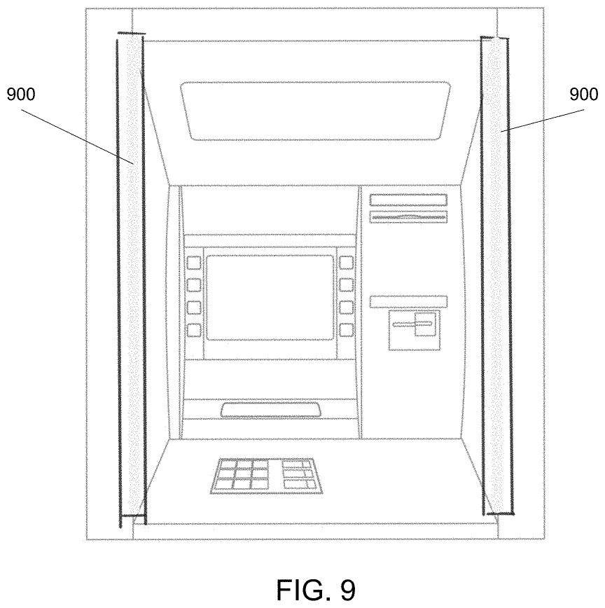

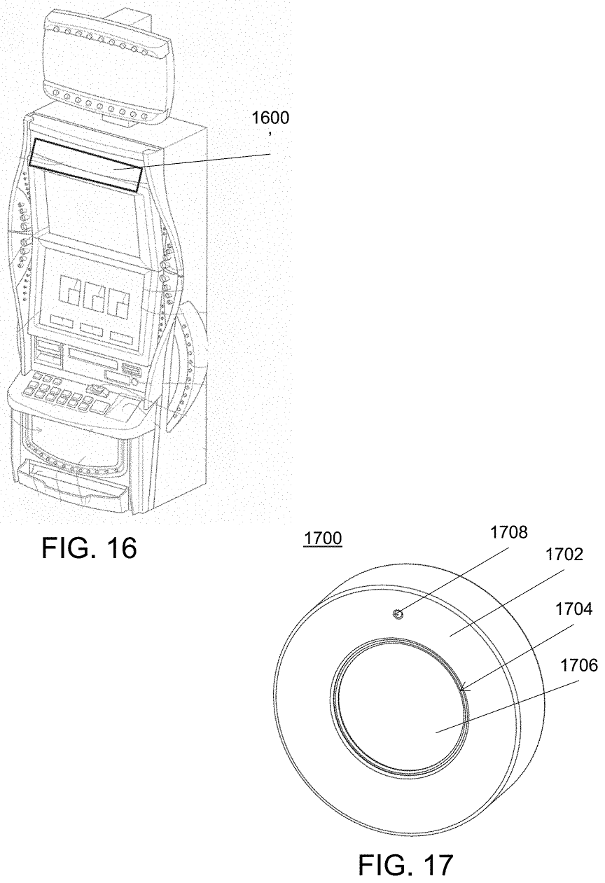

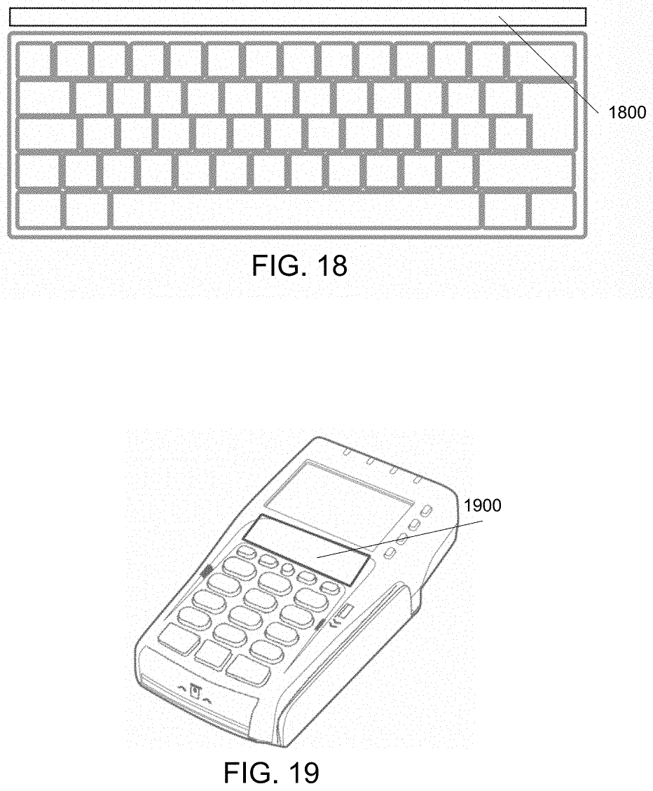

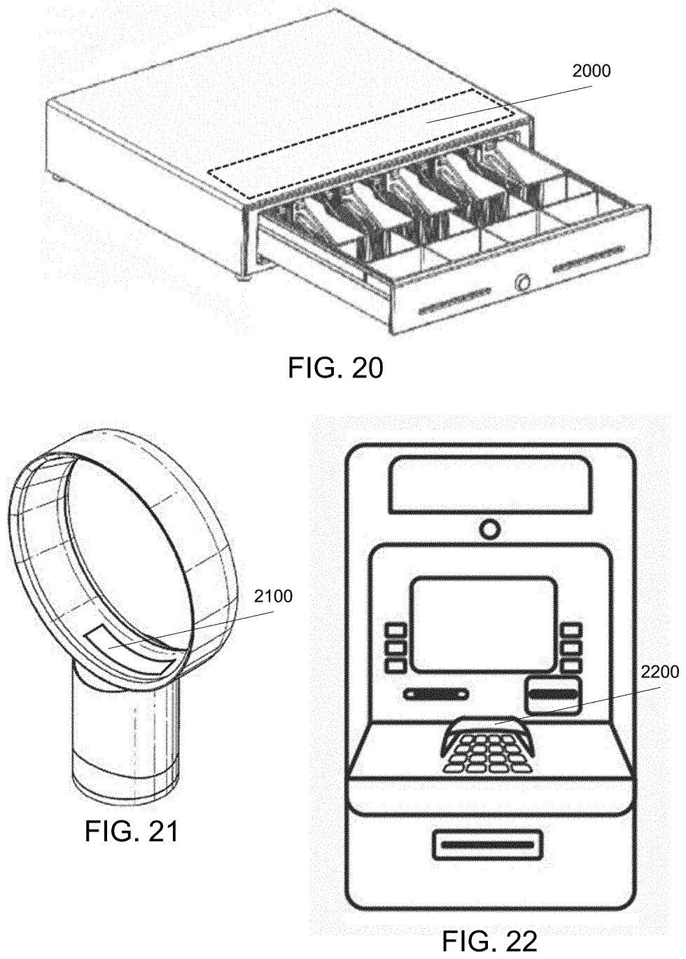

Referring now to FIG. 9, another embodiment of the invention is presented. In the present embodiment, a sterilization system 900 attached to an automated teller machine (ATM). The sterilization system 900 may be substantially similar to perform the same functions as sterilization systems described above. The sterilization system 900 may be operable to detect target pathogens on and sterilize by irradiation the surfaces and environment immediately surrounding the ATM as described above. While an ATM is shown, it is contemplated and included within the scope of the invention that the sterilization system 900 may be attached to any type of user device, such as ordering kiosks, vending kiosks, point of sale systems, tablet stands and mounts, gas pumps (1500 as shown in FIG. 15), gaming devices, such as gambling devices, including slot machines (1600 as shown in FIG. 16), keyboards (1800 as shown in FIG. 18), keypads (1900 as shown in FIG. 19), computer mice, cash drawers (2000 as shown in FIG. 20), cash registers, fans (2100 as shown in FIG. 21), card shuffling devices, device charging containers, mail delivery systems, and the like. In some embodiments, one or more of the surfaces of the ATM may be coated with a substance that facilitates sterilization thereof by UV radiation, such as, for example, titanium dioxide. Particularly, those surfaces that are most frequently touched by a user (e.g. keypad, touchscreen, item dispensers) may be so coated with titanium dioxide and/or a compound comprising the same. An alternative embodiment is shown in FIG. 22, with a sterilization system 2200 being positioned to irradiate a keypad of the ATM.

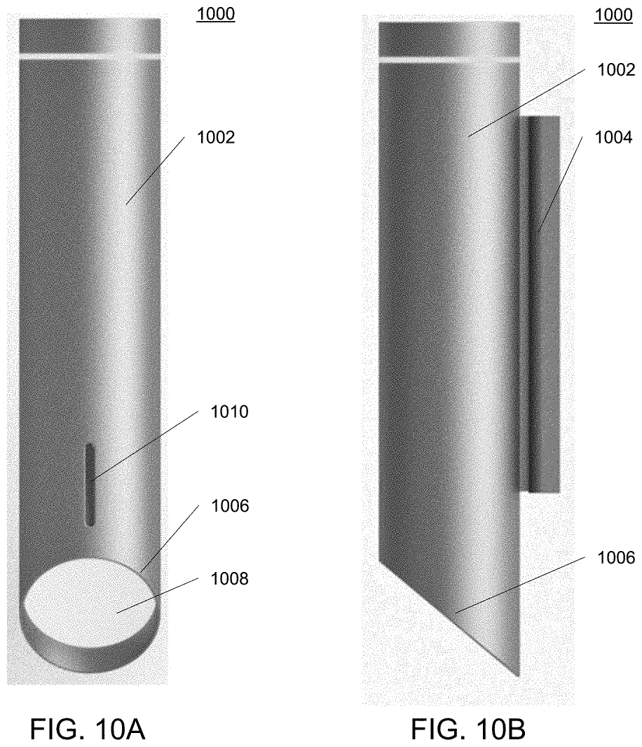

Referring now to FIGS. 10A-B, a sterilization system 1000 according to an embodiment of the invention is presented. The sterilization system 1000 may be substantially similar to the sterilization system 100 recited above, with the ability and necessary components to detect and quantify target pathogen presence within an irradiation field of the sterilization system 1000, emit sterilizing radiation, and transmit the detection and quantity of the target pathogen to a pathogen tracking system. In the present embodiment, the sterilization system 1000 comprises a housing 1002 with a surface attachment section 1004 configured to attach the sterilization system 1000 to a surface, such as a wall or stand, by any means or method known in the art, including, but not limited to, glues, adhesives, fasteners, slots, interference fits, magnetic attachment, and the like. The housing 1002 may be generally cylindrical with a tapered emitting end 1006 and an optic 1008 at the emitting end. The cylindrical geometry is exemplary only; any geometric configuration of the housing is contemplated and included within the scope of the invention. The optic 1008 may be configured to protect radiation-emitting devices comprised by the sterilization system 1000 from the environment and, in some embodiments, to diffuse, refract, collimate, or otherwise alter the emission properties of electromagnetic radiation from the sterilization system 1000.

In some embodiments, sensors comprised by the sterilization system 1000 may rely on sensing within the irradiation field via the optic 1008. In some embodiments, the housing 1002 may comprise a sensor aperture 1010 that may enable sensors comprised by the sterilization system 1000 to sense within the irradiation field.

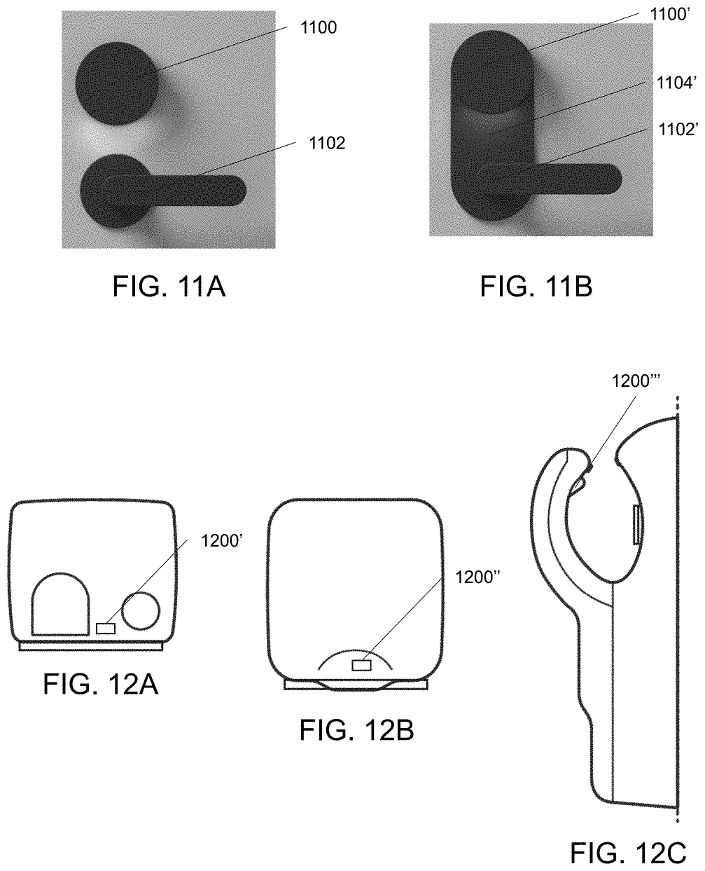

Referring now to FIGS. 11A-B, a sterilization system 1100 configured to sterilize a door handle is presented. The sterilization system 1100 may be substantially identical in componentry and function to the sterilization system 100 described above and positioned such that an irradiation field thereof may sterilize the surface of a door handle 1102. The sterilization system 1100 may be operable to detect the presence and quantity of a target pathogen on the surface of the door handle 1102 and/or an object within the irradiation field and emit a sterilizing radiations responsive thereto. In some embodiments, the sterilization system 1100 and the door handle may be disconnected. In some embodiments, the sterilization system 1100′ and the door handle 1102′ may be connected by a connecting member 1104′, which may facilitate installation and ensure proper placement of the sterilization system 100′ relative to the door handle 1102′.

Referring now to FIGS. 12A-C, sterilization systems 1200′, 1200″, 1200″′ that may be substantially identical in componentry and function to the sterilization system 100 described above, may be integrated with hand drying devices as are known in the art. The sterilization systems 1200′, 1200″, 1200″′ may be positioned to irradiate hands of users and be activated when the hand drying device is activated. Moreover, sterilization system 1200″′ may irradiate surfaces of the hand drying device in FIG. 12C that may receive substances blown off the user's hands.

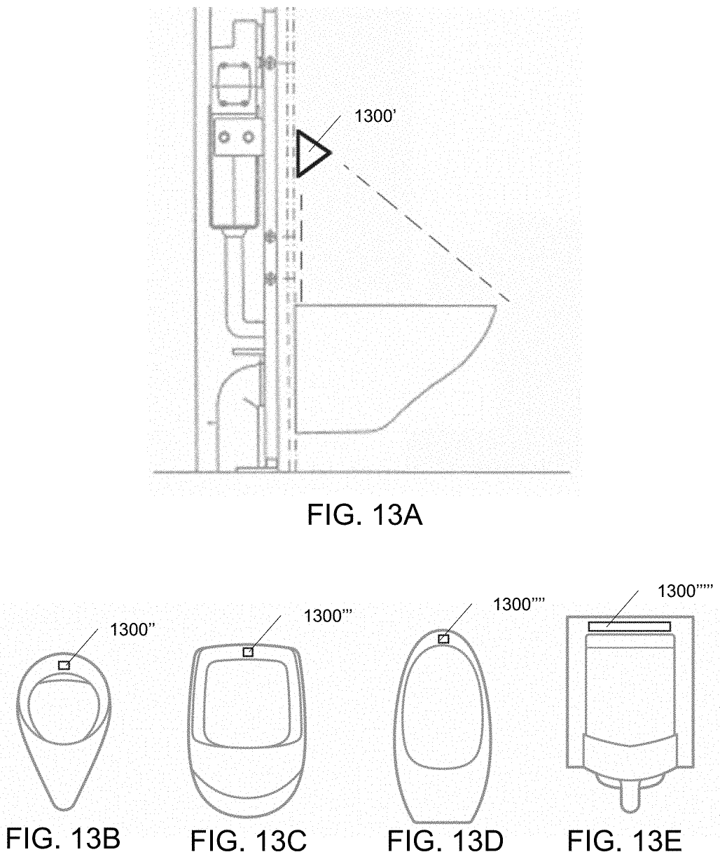

Referring now to FIGS. 13A-E, sterilization systems 1300′, 1300″, 1300″′, 1300″″, 1300″″′ that have been incorporated into human sanitation facilities. The sterilization systems 1300′, 1300″, 1300″′, 1300″″, 1300″″′ may be configured to irradiate surfaces of sanitation facilities used for human waste disposal to remove target pathogens from the sanitation facilities as described above. The sterilization systems 1300′, 1300″, 1300″′, 1300″″, 1300″″′ may be integrated with any sanitation facility as is known in the art, including toilets, urinals, bidets, commodes, and the like.

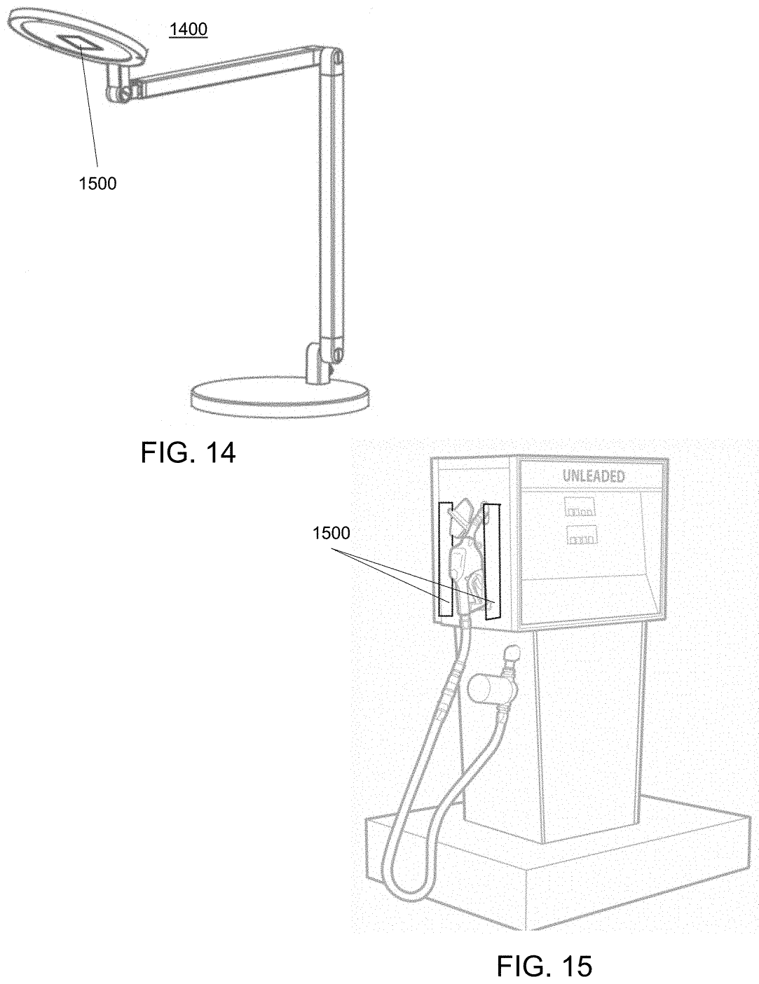

Referring now to FIG. 14, a sterilization system 1400 is presented. The sterilization system 1400 is integrate with a task light, as is known in the art. The sterilization system 1400 may include sensors and radiation-emitting devices, as well as the controlling and communicating componentry, as described above to detect and quantify target pathogens within an irradiation field thereof. The sterilization system 1400 may be operable to sterilize surfaces illuminated by the task light as well as the air between the sterilization system 1400 and the surface being illuminated. Moreover, the sterilization system 1400 may be configured to emit light perceived by users as a first color to indicate the detection of the target pathogen, emit light perceived by users as a second color to indicate the emission of sterilizing radiation, and emit light perceived by uses as a third color to indicate successful sterilization. Accordingly, the sterilization system 1400 may comprise light-emitting devices, such as LEDs, configured to emit light within different wavelength ranges to produce these different colors. While the sterilization system 1400 of the present embodiment is incorporated into a task light, it is contemplated and included within the scope of the invention that sterilization system 1400 may be incorporated into any lighting device as is known in the art, including, but not limited to, standard light bulbs, downlights, floodlights, and lighting used in illumination of large area, such as airports, bus stations, subway stations, train stations, halls, lobbies, foyers, ballrooms, trains, buses, airplanes, and the like.

Referring now to FIG. 17, a sterilization system 1700 according to an embodiment of the invention is presented. The sterilization system 1700 may include sensors and radiation-emitting devices, as well as the controlling and communicating componentry, as described above to detect and quantify target pathogens within an irradiation field thereof. The sterilization system 1700 may further comprise a housing 1702 configured to carry the componentry of the sterilization system 1700. The housing 1702 may be in any geometry, and in the present embodiments is generally disk-shaped. The housing 1702 may define an emitting aperture 1704 which may comprise an optic 1706 similar to the optic 1008 of FIGS. 10A-B. In some embodiments, sensors comprised by the sterilization system 1700 may rely on sensing within the irradiation field via the optic 1706. In some embodiments, the housing 1702 may comprise a sensor aperture 1708 that may enable sensors comprised by the sterilization system 1700 to sense within the irradiation field. The sterilization system 1700 may comprise a battery operable to provide electrical power to the componentry of the sterilization system 1700. All batteries as are known in the art are contemplated and included within the scope of the invention, included rechargeable and non-rechargeable batteries. Furthermore, the sterilization system 1700 may comprise a user input device operable to receive a user input to control the operation of the sterilization system 1700, including buttons, toggles, switches, touchscreens, and the like.

The housing 1702 may further be configured to permit the sterilization system 1700 to be positioned to sterilizing a target area. In some embodiments, the housing 1702 may comprise an attachment feature on one or more surfaces thereof, including a rear surface that is opposite the surface of the housing 1702 comprising the emitting aperture 1704. Any attachment feature as is known in the art is contemplated and included within the scope of the invention, including, but not limited to, magnets, adhesives, hooks, hook-and-loop materials, friction-increasing materials and structures, suction cups, and the like.

While the exemplary sterilization system 1700 is presented as an attachable disk-like device, it is contemplated and included within the scope of the invention that other form factors may be comprised by the sterilization system 1700, including, but not limited to, keyring-sized form factors.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

What is claimed is:

1. A sterilizing system comprising:
a sterilization device comprising:
a housing;
a plurality of radiation-emitting devices carried by the housing, comprising:
a first radiation-emitting device configured to emit electromagnetic radiation into an irradiation field having a peak intensity wavelength within a wavelength range from 217 nm to 227 nm; and
a second radiation-emitting device configured to emit electromagnetic radiation having a peak wavelength within a visible spectrum, into the irradiation field to indicate the location of the irradiation field;
a sensor configured to:
detect the presence of an object within the irradiation field; and
measure photoluminescence of a target pathogen within the irradiation field;
a dispersing apparatus configured to disperse a photoluminescent substance into the irradiation field, the photoluminescent substance being configured to luminesce when attached to the target pathogen and irradiated by radiation emitted by the plurality of radiation-emitting devices;
an indicator device configured to indicate a ready status, a treatment progress status, and a treatment complete status; and
control circuitry coupled to each of the plurality of radiation-emitting devices, the sensor, the dispersing apparatus, and the indicator device and configured to:
operate the indicator device to indicate a ready status;
receive an indication from the sensor indicating the presence of an object within the irradiation field;
operate the second radiation-emitting device to indicate the location of the irradiation field;
operate the dispersing apparatus to disperse the photoluminescent substance within the irradiation field responsive to the indication indicating the presence of an object within the irradiation field;
operate the first radiation-emitting device a first time to emit radiation;
receive an indication from the sensor indicating the contamination level of the object within the irradiation field;
determine an radiation dosage responsive to the indication of the contamination level of the object within the irradiation field;
operate the first radiation-emitting device to emit radiation to deliver the determined radiation dosage;
operate the indicator device to indicate the treatment progress status as the first radiation-emitting device operates; and
upon the first radiation-emitting device emitting the determined radiation dosage, operate the indicator device to indicate the treatment complete status;
a communication device coupled to the control circuitry and configured to communicate across a network and transmit presence and contamination level information from the control circuitry across the network;
a server positioned in communication with the communication device across a network and configured to receive the presence and contamination level information, the server comprising:
a sterilization device database configured to store individually identifiable information about the sterilization device; and
a pathogen detection database configured to store target pathogen detection and contamination level information received from the sterilization device;
wherein the server is configured to generate real-time information about the geographic distribution of the target pathogen responsive to the target pathogen detection and contamination level information stored in the pathogen detection database.
